(19) United States
(12) Patent Application Publication
(10) Pub. No.: US 2017/0021120 A1
(43) Pub. Date: Jan. 26, 2017

(54) SYSTEMS, DEVICES, AND METHODS FOR AUTOMATED ENDOTRACHEAL SUCTIONING

(71) Applicant: THE ADMINISTRATORS OF THE TULANE EDUCATIONAL FUND, New Orleans, LA (US)

(72) Inventors: Daniel ASH, New Orleans, LA (US); Caroline EDWARDS, Menlo Park, CA (US); Stephen LEE, Evanston, IL (US); Connor MACCRIMMON, New Orleans, LA (US); Dylan Sylvestor, Albuquerque, NM (US); George TORSILIERI, New Orleans, LA (US); Donald P GAVER, New Orleans, LA (US)

(73) Assignee: THE ADMINISTRATORS OF THE TULANE EDUCATIONAL FUND, New Orleans, LA (US)

(21) Appl. No.: 15/302,447

(22) PCT Filed: Apr. 7, 2015
(86) PCT No.: PCT/US15/24752

§ 371 Date: Oct. 6, 2016

Related U.S. Application Data
(60) Provisional application No. 61/976,394, filed on Apr. 7, 2014.

(51) Int. Cl. A61M 16/04; A61M 16/08

(52) U.S. Cl. CPC A61M 16/0463; A61M 16/0816; A61M 16/0465; A61M 2205/10; A61M 2205/52; A61M 2205/502

(57) ABSTRACT

The present disclosure provides systems, devices and methods for automated endotracheal suctioning The device comprises an adaptor, which in some embodiments retrofits into existing suctioning equipment. The system includes the adaptor as well as suctioning tube, componentry for automatically deploying and retracting the suctioning tube, suctioning device, and processor to manage automation of deployment/retraction of the tube and suctioning of fluid. The method includes use of the device to automatically suction fluids and may also include programming the device to take into consideration safety concerns such as overuse or suction tube placement.

### CROSS-REFERENCES TO RELATED APPLICATIONS

This application claims the benefit of priority to U.S. Provisional Application No 61/976,394, filed Apr. 7, 2014, which application is incorporated herein by reference in its entirety.

### FIELD

The present specification relates to systems, devices, and methods for endotracheal suctioning. The specification also relates to systems, devices and methods for automated endotracheal suctioning, including computer-implemented systems, devices and methods for automated endotracheal suctioning.

### BACKGROUND

Patients on long-term ventilation support arc at increased risk for infections, both at the insertion site of the cannula and internally. In order to maintain a clear airway, decrease infection risk and maximize respiratory function, endotracheal suctioning is a necessary procedure for patients with an artificial airway. The presence of a tracheostomy tube or endotracheal tube impairs the ability to cough and void pulmonary secretions; many patients with these tubes require suctioning to clear the airway.

A number of medical conditions may require a patient to have a tracheostomy or endotracheal tube inserted. Many of these conditions also involve loss of motor function, which means that assistance from a caregiver is needed to suction the airway. The suctioning procedure involves inserting a catheter or other tube into the tracheostomy or endotracheal tube and applying suction, usually with a vacuum, then removing the suction tube from the tracheostomy or endotracheal tube. Depending on various factors, including the amount of secretions produced, a patient may requiring airway suctioning nine to twelve times per day or more. For those patients with loss of motor function, frequent suctioning necessitates the near-constant presence of a caregiver. This imposes a high burden on the caregiver, compounded by interrupted sleep due to the need to suction several times overnight.

Several technologies exist to aid with suctioning, though none allow a patient to be more independent. One available suctioning aid involves an attachable piece or pieces to collect the fluid secretions. Others involve a method of “chest percussion”, wherein a vibrating plate is placed on the chest of the patient to rattle the fluids to loosen them and be less disturbing to the patient. However, none of these technologies allow the patient to be more autonomous or independent from their caregiver, nor do they reduce the patient's need for suctioning There is thus a need for a system to allow for more autonomous airway suctioning

### SUMMARY

A number of issues are present in each of the currently-available suctioning technologies. There is a need for a device to alleviate these issues, for example to increase patient autonomy and reduce the burden on caregivers, while providing suctioning at least as effectively as current methods. The present specification discloses a device and system, which in some embodiments allows for automated suctioning of a patient's airway. The present specification also discloses a method of suctioning a patient's airway using an automated system. In some embodiments, the disclosed system and method provide a means of effective airway suctioning that allows a patient to live more independently from his or her caregiver than the currently-available suctioning technologies described above.

In some embodiments, the automated implementations of the devices, systems and methods of this disclosure take into account one or more of the following patient safety and/or comfort considerations: The device is easily detachable to prevent the pulling of the trachea. The device and system are lightweight for patient comfort and to prevent damage to the trachea. Both the device and system are at least as durable as current ventilator tubing. The automated suctioning system requires the same or less maintenance as the current suctioning process while effectively avoiding contamination. The automated suctioning system is safe for the patient and user-friendly. The entire procedure involves comparable or less time than the current process, and little room for error. The process is consistent without involving the caregiver.

In some embodiments, the device comprises an adaptor having a first open-ended port comprising a proximal end configured to connect to a cannula and a distal end configured to connect to a ventilator tube and a second open-ended port for receiving a suctioning tube and guiding the suctioning tube into the cannula. In some embodiments, the adaptor is a universal adaptor compatible with existing ventilator systems. In some embodiments, the adaptor includes componentry for automated engagement and disengagement of a suctioning tube, including automated activation and deactivation of a suctioning system. The componentry may include a motor or piston-rod or other mechanism for driving the feeding of a suction tube into or away from a cannula. The componentry may also include a computer processing unit (“CPU”) and instructions, which if executed by the CPU result in engaging or disengaging the suctioning tube and activating or deactivating the suctioning system.

In some embodiments, the system comprises an adaptor as described above, a suctioning tube, a suctioning device such as a vacuum, a suction tube positioning device for automated engagement and disengagement of the suctioning tube into a cannula, a mechanism for activating the positioning device, and a mechanism for deactivating the positioning device, which may be the same as the activating mechanism.

In some embodiments, the method for automated suctioning of a patient's airway includes: activating a positioning system, deploying a tube to a pre-determined depth using the positioning system, activating a suctioning means for a predetermined period of time, deactivating the suctioning means following the expiration of the predetermined period of time, activating the positioning system and removing the tube using the positioning system.

In some embodiments, the disclosure provides a system that delivers effective and safe self-directed airway suctioning In some or further embodiments, the disclosure provides a method of airway suctioning using a mechanical device, such as a motor or pneumatics, deployed by the user.

The identified embodiments are exemplary only and are therefore non-limiting. The details of one or more non-limiting embodiments of the invention are set forth in the accompanying drawings and the descriptions below. Other embodiments of the invention should be apparent to those of ordinary skill in the art after consideration of the present disclosure.

While certain novel features of this invention shown and described below are pointed out in the annexed claims, the invention is not intended to be limited to the details specified, since a person of ordinary skill in the relevant art will understand that various omissions, modifications, substitutions and changes in the forms and details of the invention illustrated and in its operation may be made without departing in any way from the spirit of the present invention. No feature of the invention is critical or essential unless it is expressly stated as being “critical” or “essential.

### BRIEF DESCRIPTION OF DRAWINGS

The following drawings form part of the present specification and are included to further demonstrate certain aspects of one or more embodiments of the present invention. Embodiments of the invention may be better understood by reference to one or more of these drawings in combination with the description of specific embodiments presented herein.

FIG. 1 shows an illustration of the manual suctioning method.

FIG. 2 shows a picture of the equipment currently used in airway suctioning

FIG. 3 shows a flow chart for an embodiment of a method for automated suctioning in accordance with this disclosure.

FIG. 4a is a stylized perspective view of the adaptor component of the automated suction system.

FIG. 4b is a stylized side view of the adaptor component of FIG. 4a.

FIG. 5a is a side perspective view of another embodiment of the adaptor device, in this case using rollers to insert and withdraw a suctioning tube.

FIG. 5b is a transparent side view of the embodiment of the adaptor device shown in FIG. 5a.

FIG. 6 shows a side perspective view of an embodiment of the adaptor device with rollers.

FIG. 7 shows a top perspective view of the adaptor device embodiment of FIG. 6.

FIGS. 8a ad 8b are stylized side and top views, respectively, of one embodiment of a drive mechanism, in this case an embodiment of a motor drive mechanism.

FIG. 9 shows one embodiment of the roller.

FIG. 10 is a perspective view of the adaptor of FIG. 6 in use to deploy and retract a suction tube.

FIG. 11 is an illustration of an embodiment of an automated suctioning system according to this disclosure fitted to a patient.

### DETAILED DESCRIPTION

Detailed descriptions of one or more embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

Wherever any of the phrases “for example,” “such as,” “including” and the like are used herein, the phrase “and without limitation” is understood to follow unless explicitly stated otherwise. Similarly “an example,” “exemplary” and the like are understood to be non-limiting.

The term “substantially” allows for deviations from the descriptor that do not negatively impact the intended purpose. Descriptive terms are understood to be modified by the term “substantially” even if the word “substantially” is not explicitly recited. Therefore, for example, the phrase “wherein the lever extends vertically” means “wherein the lever extends substantially vertically” so long as a precise vertical arrangement is not necessary for the lever to perform its function.

The terms “comprising” and “including” and “having” and “involving” (and similarly “comprises”, “includes,” “has,” and “involves”) and the like are used interchangeably and have the same meaning Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

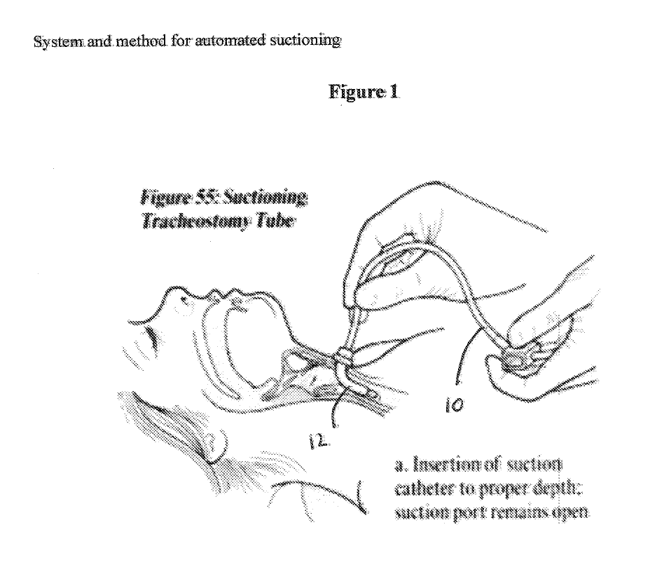

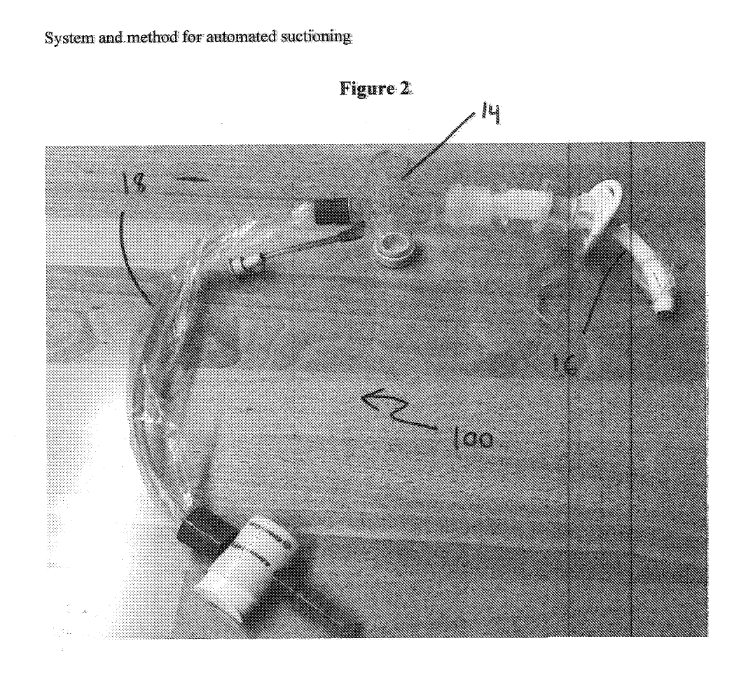

Referring now to the Figures, where like numbers reflect like elements, FIG. 1 shows an illustration of the current suctioning method, which is done manually. The caregiver inserts a tube 10 connected to a vacuum (not shown) or other suctioning device into the airway tube 12 and then applies suction for a period of time. After the suctioning is complete, the caregiver pulls the suctioning tube out of the airway tube. FIG. 2 shows a picture of the equipment used in the current endotracheal suctioning system 100, including the adaptor piece 14 that is currently used to connect the tracheostomy tube 16 to the ventilator 18.

In developing the devices, systems and methods consistent with this disclosure, consideration was give to designing an automated system which is user-friendly in that it can be installed with no change to the patient's current intubation, as illustrated in FIGS. 1 and 2.

Consideration was also given to ensuring that the suctioning tube is inserted to a specific distance and is not inserted too far or not far enough, and that the suction is consistently applied for a set period of time. Other design considerations include avoiding damage to the trachea, preventing irritation at the insertion site, and reducing patient infection. Certain embodiments within the scope of this disclosure take into account one, some or all of these considerations.

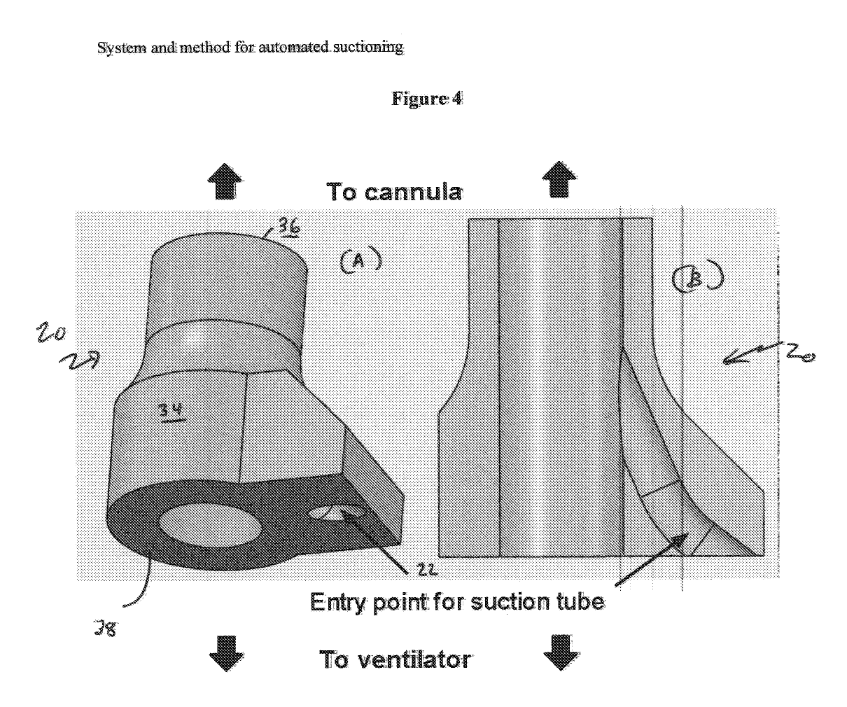

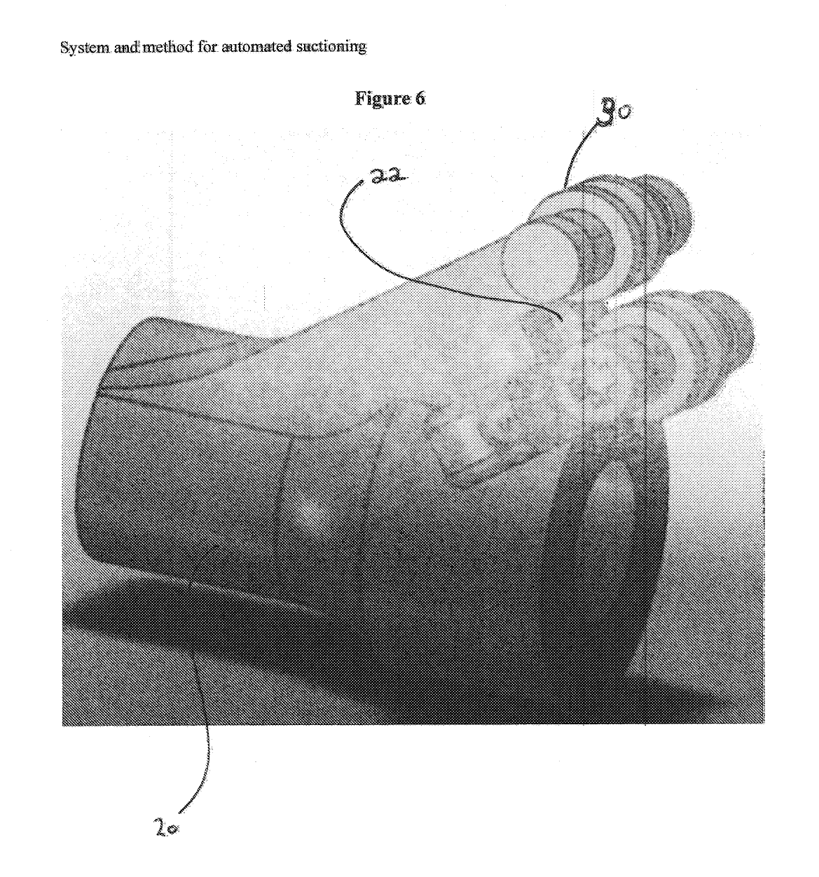

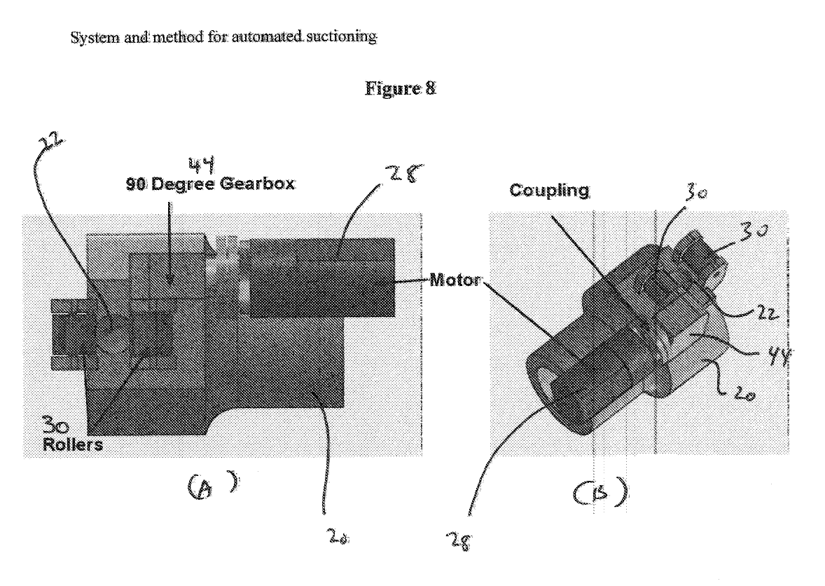

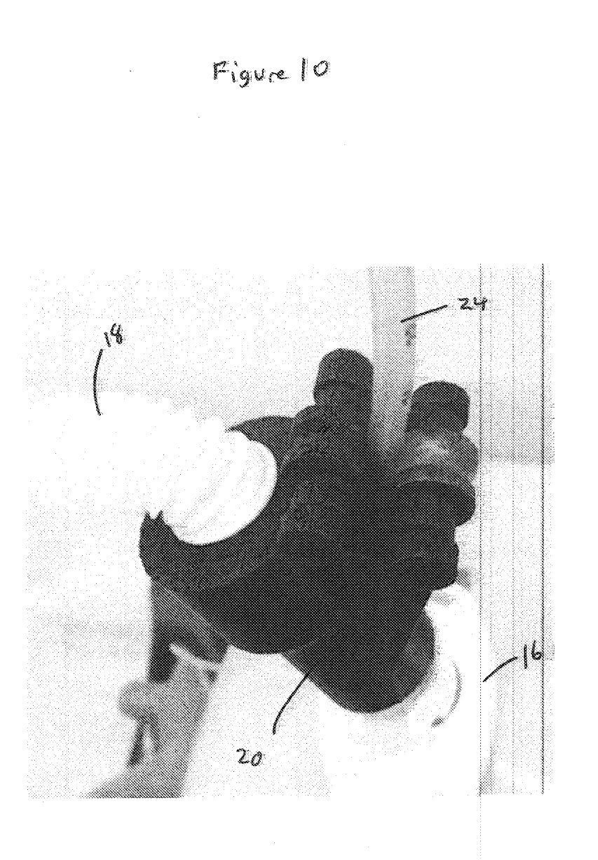

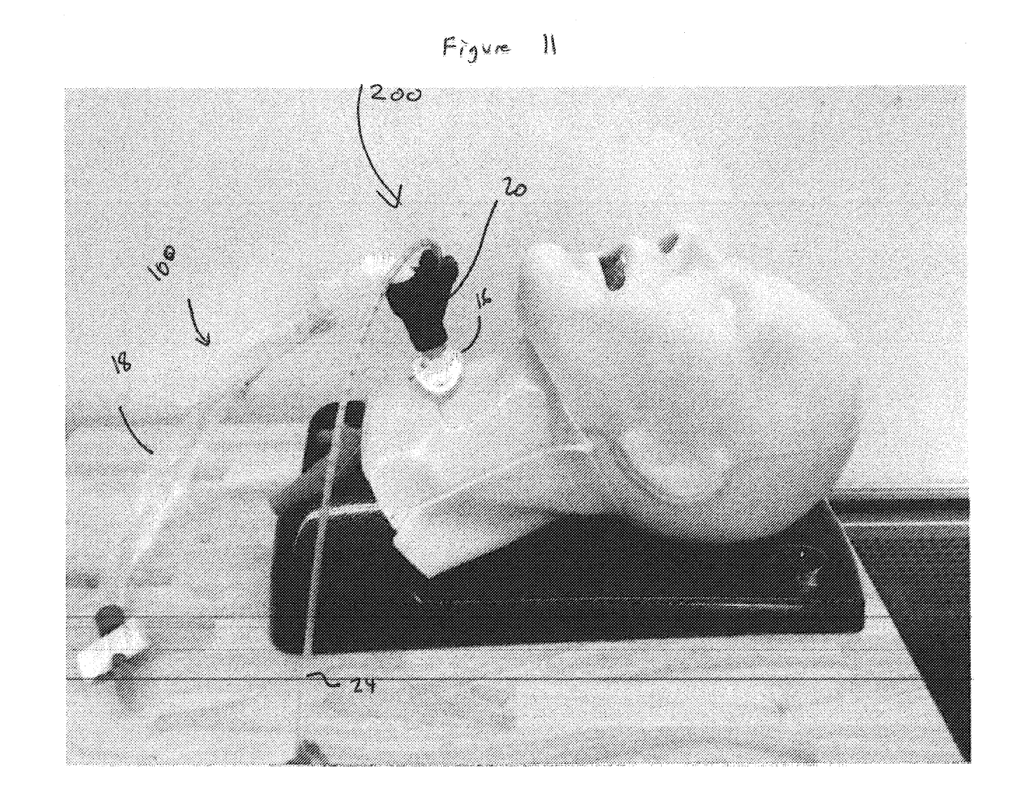

For example, certain embodiments within scope of this disclosure include a universal adaptor 20 (see, e.g., FIGS. 4-6), which is designed to substitute for the current adaptor piece 14 such that it can be used with current ventilators. In further or other embodiments, the universal adaptor 20 includes an open-ended (i.e. open at both the distal and proximal end) port 22 to provide an access point for a suction tube 24 (see FIGS. 10 and 11) to be inserted into a tracheostomy or endotracheal tube 16 (see FIG. 10). And, as shown in FIG. 8, in some embodiments, a motor 28 attached to the connector 20 feeds the suction tube 24 via one or more rollers 30 into the tracheostomy or endotracheal tube 16. A vacuum is also automatically activated to provide suction for a preset period of time. In some embodiments, the vacuum is activated when the motor is activated to deploy the suction tube 24. In some embodiments, the vacuum is activated after the suction tube 24 has been deployed and the motor has been shut off. In one embodiment, the suctioning tube is a catheter. In some embodiments, a stopper, ring, edge or other appropriate means may be attached to the suctioning tube to prevent it from descending past a predetermined depth. Once the preset period of time is completed, the vacuum automatically shuts off and is retracted through the one or more rollers 30 via the motor 28. In some embodiments, a button (not shown) is used to activate the motor. In other embodiments, the motor is activated via eye tracking equipment (not shown).

In some embodiments, the automated device is characterized by one or more of: being at least as durable as the current equipment, requiring a similar or less amount of maintenance, providing effective (similar) suctioning in the same amount of time or less than the current procedure, and leaving little room for error.

FIG. 11 illustrates an embodiment of an automated endotracheal suctioning system 200 according to this disclosure in use on a patient and integrated with the current, manual suctioning system 100. In one embodiment, the present disclosure provides an automated suctioning system that uses a connector device inserted between the ventilator tube and the tracheostomy cannula of a current system. As shown, the current system 100 includes the tracheostomy tube 16 and ventilator 18. However, the current adaptor 14 has been replaced with a universal adaptor 20 in accordance with this disclosure, which universal adaptor 20 enables the use of a second suctioning tube 24 for automated suctioning Accordingly, in some embodiments, the automated suctioning system 200 comprises: the universal adaptor 20; the second suctioning tube 24; a suctioning system such as a vacuum (not shown); cannula 16; and, electronics (not shown) for driving the automated system 200.

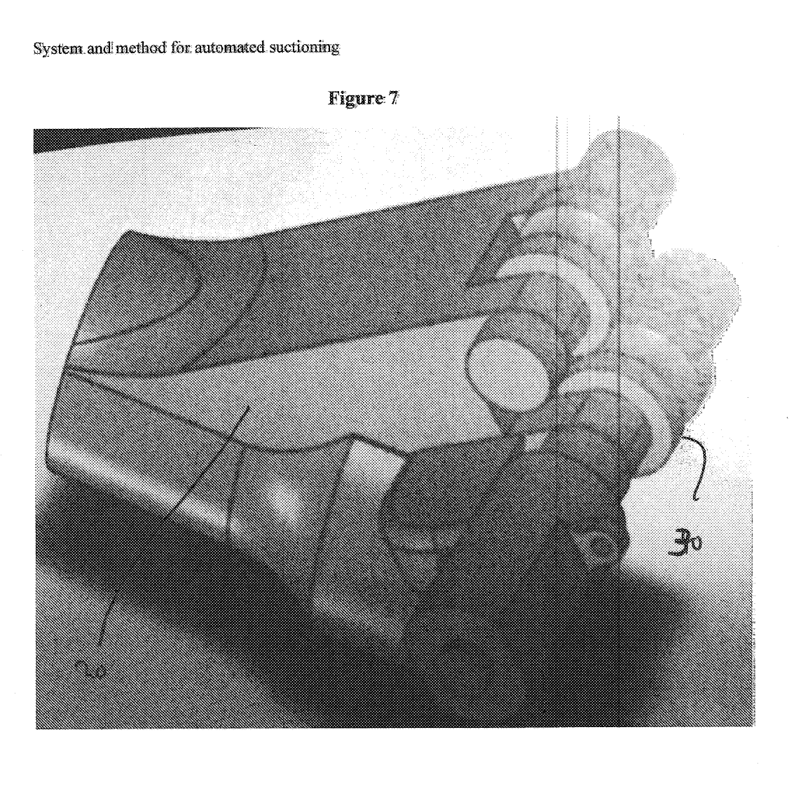

FIGS. 4-11 show embodiments of the universal adaptor 20 in greater detail. (Although the connector 20 is referred to as a “universal adaptor” this is not meant to limit the subject of this disclosure. Using a connector that can be fitted into existing ventilation equipment is a non-limiting design choice; the connector can also be manufactured for specific ventilation equipment, for example for equipment that is designed simply to operate the automated system and not necessarily have backward compatibility with existing systems or have the option of being integrated side-by-side with existing systems.)

FIGS. 4a and 4b arc perspective and side view illustrations of an embodiment of an adaptor 20 according to this disclosure. As shown, the adaptor 20 comprises a body 34 having a proximal end 36 configured to connect to a cannula (not shown in FIG. 4) and a distal end 38 configured to connect to a ventilator (not shown in FIG. 4). The body serves as an open-ended port (i.e. open on both ends) to enable exchange of air from the ventilator to the patient. The adaptor also comprises a second open-ended port 22, which receives a suction tube (not shown in FIG. 4) and guides the suction tube toward the cannula.

In some embodiments, the connector device may be comprised of medical grade polystyrene, medical grade polyvinyl chloride, medical grade polyisoprene or any other appropriate material. In one embodiment, the connector device may be hollowed out. In some embodiments, the connector device may be disposable. In some embodiments, the connector device is enclosed in a sterile bag along with the suctioning tube.

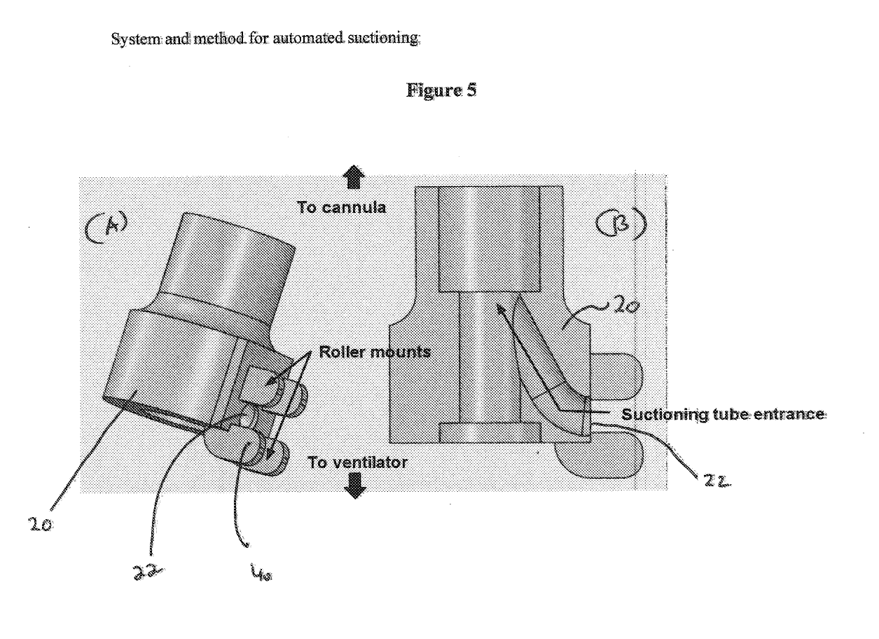

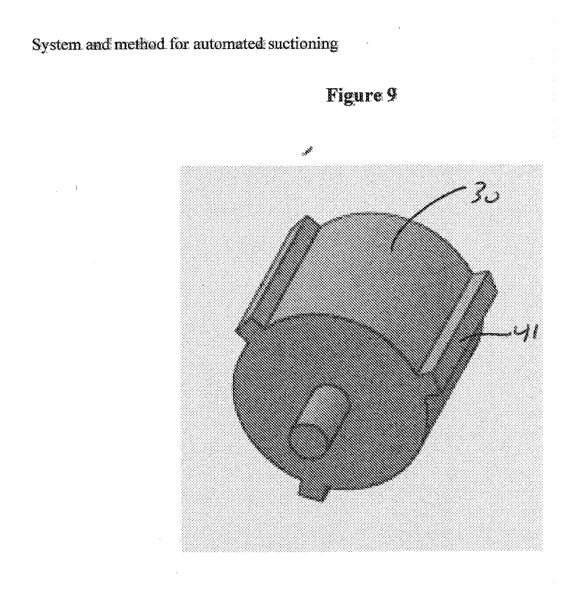

FIGS. 5-8 illustrate another embodiment of the adaptor 20. According to this embodiment, the connector device 20 includes one or more rollers 30, which may be fitted to the body 34 of the adaptor 20 by roller mounts 40, for receiving and guiding the suctioning tube through the port 22 and into the cannula. In some embodiments, the one more rollers include one fixed roller and one rotating roller connected to the one or more motors. In another embodiment, the one or more rollers may be clipped, snapped or inserted into place. The rollers may be coated in a substance to increase friction, such as latex, shrink wrap, rubber, or an aerosol spray coating. In some embodiments, one or more rollers may be textured to increase friction, such as with edges, bumps, ridges, dots, cross-hatching, waves, lines or other textures. FIG. 9 shows one embodiment of a roller 30 with edges 41. The roller may be comprised of silicone/latex rubber, medical grade polystyrene, medical grade polyvinyl chloride, polyisoprene or any other appropriate material.

In order to automate the feeding of the suction tube, a mechanism to drive the rollers to gather and feed the suction tube through the port may be used. In some embodiments, as shown in FIG. 8, the drive mechanism may be one or more motors 28. In the illustrated embodiment, the motor 28 is a gear motor, connected to a gearbox 44. In one embodiment, the motor is powered by batteries. In another embodiment, the motor is powered by a power source that also powers the vacuum and/or the ventilator. In one embodiment, the motor is powered by a power source that also powers the patient's wheelchair. As a person of skill should appreciate from reading this disclosure, the drive mechanism need not be a motor but can be any suitable device/componentry that can cause movement of the suction tube through the port into the cannula. For example a pneumatic system rather than a motorized system may be used to feed the suction tube.

The system also may include a computer processing unit or CPU and a memory device, which includes instructions for execution by the CPU, and controls operation of the system. For example, in the illustrated embodiment, the motor is connected to a CPU that controls the speed at which the motor rotates, the depth to which the suctioning tube is inserted, and the activation of the vacuum. In some embodiments, the unit is preprogrammed to move the suction tube over a given distance. In other embodiments, the depth to which the suctioning tube is inserted may be controlled by a caregiver specifying a predetermined time for the motor to be activated to deploy the tube. In one embodiment, the CPU will monitor the amount of current drawn by the motor and will shut down the motor if the drawn current exceeds a predetermined value. As another alternative, the caregiver may program in the depth to which the suctioning tube should be inserted and the CPU will compute the time the motor should operate based on the motor parameters to achieve the desired depth of insertion.

In operation, in one embodiment, the user presses a button to activate the system (in the illustrated embodiment, this activates a motor). The motor drives a roller that deploys a tube to a predetermined depth. A vacuum automatically is activated and provides suction for a preset period of time. In some embodiments, the vacuum is activated after the predetermined depth is reached. In some embodiments, the vacuum is activated at the time the motor is turned on to deploy the suction tube. In some embodiments the vacuum is activated after the motor is turned on and before the predetermined depth is reached. After the preset period of time expires, the vacuum automatically shuts off and then the motor automatically activates to drive a roller to retract the tube. In a preferred embodiment, the user is a patient with a tracheostomy tube or an endotracheal tube. In other embodiments, the automatic suction method may be used in industrial applications or in other medical or dental procedures, including surgery.

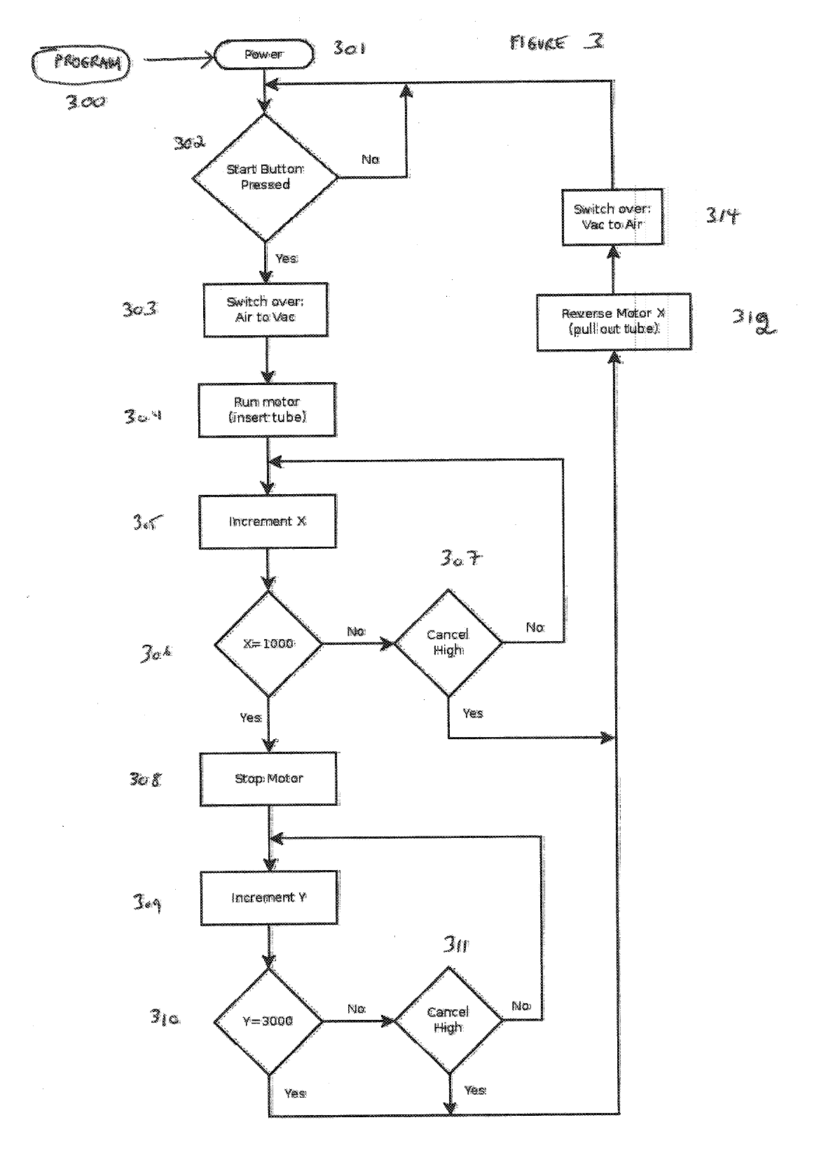

FIG. 3 illustrates an embodiment of the operation of a system according to this disclosure. As an initial step, at block 300, if the unit does not come pre-programmed or if the unit has an option to be programmed or re-programmed, a caregiver programs the device with the desired parameters. The caregiver may be, for example, physicians, clinicians, nurses or other healthcare providers. Setting the parameters may be accomplished through a computer user interface, which in one embodiment may be on the device itself or another embodiment may be viewed on a computer screen after connecting the device (for example via a USB port or wirelessly) to a computer. In some embodiments, the user programs the depth of insertion of the suctioning tube (such as the amount of tubing that should pass through the adaptor) either, for example, by providing a distance instruction or a time instruction. In some embodiments, the unit may be equipped to detect the appropriate depth and stop automatically.

One challenge for an automated suctioning system is to prevent patients from repeatedly suctioning the airway when a blockage is not present or cannot be cleared with the tube alone. Suctioning the airway too often can cause serious damage to the lungs and airway and can also cause oxygen deprivation, as permanently ventilated patients cannot be given high percent oxygen for fear that they will become dependent. In order to address this, one embodiment of the present invention provides the user an opportunity to program the maximum number of times the suctioning tube may be deployed over a specified period of time. (The unit may also come preprogrammed with this paramenter.) In some embodiments, the CPU may allow a caregiver to specify a predetermined time period during which the motor may not be activated. In an another embodiment, a means of monitoring the peak pressure is included to confirm whether or not the suctioning was successful, as the peak pressure lowers when the airway is clear of obstacles.

At block 301, power is provided to the device. In one embodiment, the automated suctioning system is activated by a button pressed by the user. In some embodiments, the automated suctioning system is activated by means of an eye tracking device. As shown at block 302, the CPU checks to see if power was successfully provided to the unit. If the unit has not turned on, the user may again attempt to activate the unit until successful. Once the device is powered up, the process moves on to block 303 in which suctioning begins causing the system to switch over from air to vacuum. In some embodiments, switching over from air to vacuum occurs only after the suctioning tube is fully in place in the patient and the motor (or other drive mechanism) has stopped (i.e. after block 308).

At block 304, the drive mechanism is turned on to cause the suction tube to be driven through the open-ended port 22 through the cannula and into the patient. Once the suction tube is in position at the desired depth, the drive mechanism stops (block 308).

Once the suction tube is in place, the unit continues to provide vacuum for a predetermined time (or in some embodiments, the system may include componentry to detect when suctioning is finished and automatically trip the vacuum to stop). After the pre-determined time is up, at block 310, the drive mechanism is turned on (for example in reverse if the same drive mechanism is used) in order to remove the suction tube from the patient, at block 312. At block 314 (which may occur simultaneously with block 312, the system switches over from vacuum to air). A user may then once again suction by activating the unit (starting the loop at block 301—unless otherwise prohibited from doing so by the programming of the unit.

In some embodiments, and as shown in FIG. 3, the automated suctioning system may include a stop button (separate from the power button to alleviate error) to halt the vacuum and retract the catheter without regard for the predetermined distance or suction runtime. In the case of malfunction, manual suction will still be possible (for example using the componentry 16, 18 shown in FIG. 11 associated with the manual suctioning system) and the suction tube may be removed from the connector device manually. In one embodiment, the rollers would break away to allow for an easy manual removal of the suction tube. The stop button may trip the system for example either during deployment of the suctioning tube as shown at block 307 or during suctioning after the tube is fully deployed at block 311.

FIG. 3 also further illustrates more detailed steps that may occur when a specific type of motor is chosen as the drive mechanism. As shown in the loop defined by blocks 304-307, if the motor is for example programmed to run for 1000 milliseconds, the system periodically checks (in the illustrated example the check occurs every millisecond) to determine the length of time the motor has been running in total (“Increment X”, block 305). When the system counts 1000 milliseconds (block 306), the motor shuts off. If the cancel button is hit any time before X=1000 milliseconds in the present example, the motor shuts off and the process proceeds to block 312 in which the motor is run in reverse to retract the suction tube and block 314 in which vacuum is switched back over to air.

Similarly in the illustrated embodiment, a loop is built in during suctioning after the tube is in position. As shown by blocks 309-311, the system has been programmed to suction for 3 seconds (3000 milliseconds), vacuum is cancelled and checks every millisecond to determine how many millisecond total the vacuum has been running If the cancel button is hit anytime in this loop before Y reaches 3000 milliseconds, the process moves forward to blocks 312 and 314.

A number of embodiments have been described but a person of skill understands that still other embodiments are encompassed by this disclosure. It is understood, therefore, that this disclosure and the inventive concepts are not limited to the particular embodiments disclosed, but are intended to cover modifications within the spirit and scope of the inventive concepts including as defined in the appended claims. Accordingly, the foregoing description of various embodiments does not necessarily imply exclusion. For example, “some” embodiments or “other” embodiments may include all or part of “some”, “other,” “further,” and “certain” embodiments within the scope of this invention. Methods and devices within the scope of the disclosure can also be defined in accordance with the below embodiments. Non-limiting methods and devices within the scope of the disclosure can also be defined in accordance with the below embodiments.

1. An automated suctioning system comprising:

a. one or more connector devices;

b. one or more tubes;

c. one or more means of deploying the one or more tubes;

d. at least one suctioning device; and

e. one or more means of activating the one or more means of deploying the one or more tubes.

2. The device of embodiment 1, wherein the one or more means of deploying the one or more tubes comprises at least one roller.

1. The device of embodiment 2, further comprising at least one motor connected to the at least one roller.

2. The device of embodiment 1, wherein the at least one suctioning device comprises a vacuum.

3. The device of embodiment 1, wherein the one or more means of activating the one or more means of deploying the one or more tubes comprises a button.

4. The device of embodiment 1, wherein the one or more means of activating the one or more means of deploying the one or more tubes comprises eye tracking equipment.

5. The device of embodiment 1, further comprising a computer processing unit.

6. The device of embodiment 3, further comprising a computer processing unit and software to control the at least one motor.

7. The device of embodiment 3, further comprising a gear box connected to the motor.

8. The device of embodiment 2, wherein the at least one rollers consist of one fixed roller and one free-spinning roller.

9. The device of embodiment 10, further comprising a motor connected to the free-spinning roller.

10. The device of embodiment 2, wherein the at least one rollers are textured.

11. The device of embodiment 12, wherein the texture on the at least one roller is selected from the group comprising bumps, edges, ridges, dots, cross hatching, waves or lines.

12. The device of embodiment 2, wherein the at least one rollers are coated in a substance to increase friction.

13. The device of embodiment 14, wherein the substance to increase friction is selected from the group comprising rubber, latex, shrink wrap, aerosol coating or spray coating.

14. The device of embodiment 3, wherein the at least one motor comprises a gear motor.

15. The device of embodiment 5, further comprising a second button to stop activating the activating the one or more means of deploying the one or more tubes.

16. A method for automated suctioning comprising:
a. activating a motor;
b. deploying a tube to a predetermined depth via said motor;
c. activating a suctioning means for a preset period of time;
d. deactivating said suctioning means following the expiration of said preset period of time;
e. activating said motor; and
f. removing said tube via said motor.

17. The method of embodiment 17, wherein the motor is activated via one or more buttons.

18. The method of embodiment 17, wherein the motor is activated via eye tracking equipment.

19. The method of embodiment 17, wherein the suctioning means comprises a vacuum.

20. The method of embodiment 17, wherein the tube comprises a catheter.

21. The device of embodiment 1, wherein the connector is comprised of a material selected from the group comprising medical grade polystyrene, medical grade polyvinyl chloride and medical grade polyisoprene.

22. The device of embodiment 2, wherein the one or more rollers are comprised of a material selected from the group comprising silicone rubber, latex rubber, medical grade polystyrene, medical grade polyvinyl chloride or polyisoprene.

23. The device of embodiment 1, wherein the one or more tubes comprises a catheter.

24. A device comprising: an adaptor comprising: a first open-ended port having a proximal end configured to connect to a cannula and a distal end configured to connect to a ventilator tube; and, a second open-ended port for receiving a suctioning tube and guiding the suctioning tube into the cannula.

25. A device according to embodiment 24, wherein the adaptor is a universal adaptor compatible with existing ventilator systems.

26. A device according to embodiment 24 or 25, wherein the adaptor further comprises componentry for automated engagement and disengagement of the suctioning tube.

27. A device according to embodiment 26, further comprising a positioning system for positioning the suctioning tube by either deploying the suctioning tube into the cannula, removing it from the cannula, or both.

28. A device according to embodiment 27, wherein the positioning system is a motorized system that deploys the suctioning tube from a start position to an end position, which is in or though the cannula and that also retracts the suctioning tube to the start position.

29. A device according to embodiment 27, wherein the positioning system is a pneumatic system that deploys the suctioning tube from a start position to an end position, which is in or though the cannula and that also retracts the suctioning tube to the start position.

30. A system, comprising: a device according to any of embodiments 3-6; and, a mechanism for activating the suctioning tube positioning system.

31. A system according to embodiment 30, further comprising a suctioning tube and a suctioning device, which device creates suction in the suctioning tube when engaged.

32. A system according to embodiment 31, wherein the activation mechanism results in deploying the suctioning tube, activating suction, and thereafter removing the suctioning tube from the tracheostomy cannula.

33. A system according to embodiment 32, further comprising a mechanism for deactivating the activating mechanism to cancel deployment of the suctioning tube, suctioning, or both.

34. A system according to embodiment 33, wherein the deactivating mechanism further results in removing the suctioning tube from the cannula if it has been deployed in the cannula.

35. A system according to any of embodiments 30-34, further comprising a computer processing unit (“CPU”); a memory containing instructions for execution by the processor, which if executed results in automated control of the activation mechanism; and, a client user interface for triggering the processor to execute the instructions.

36. A system according to embodiment 35, wherein the client user interface comprises a button, which when depressed results in the processor executing the instructions.

37. A system according to embodiment 26, wherein the instructions result in: activating the positioning system to deploy the suctioning tube into or through the cannula; activating the suctioning system to suction for an amount of time or until an amount of liquid is collected; and, activating the positioning system to remove the suctioning tube.

38. A system according to embodiment 37 wherein the positioning system deploys the suctioning tube by moving the suctioning tube in a forward direction through the port a predetermined distance or for a predetermined time, and removes the suctioning tube by moving the suctioning tube in a reverse direction for the same predetermined distance or predetermined time.

39. A system according to embodiment 37 or 38 wherein the suctioning system is activated for a predetermined amount of time or until a predetermined volume is collected.

40. A system according to any of embodiments 37-39, further comprising a cancel button for shutting down deployment, suctioning or both.

41. A system according to embodiment 40, wherein depressing the cancel button further results in retracting the suctioning tube to the start position.

42. A system according to any of embodiments 30-34, wherein the system further comprises a computer program product for operating the suctioning system, comprising: a tangible computer readable storage medium having a computer readable program code embedded therein, the computer readable program code configured to: activate and deactivate the positioning system and activate and deactivate the suctioning system.

43. A system according to embodiment 42, wherein the positioning system is a motorized system and wherein activating and deactivating the positioning system and activating and deactivating the suctioning system comprises, when a start instruction is received, activating a vacuum and switching from air, running a motor for a predetermined period of time sufficient to deploy the suction tube unless a cancel instruction is received, thereafter operating or continuing to operate the vacuum for a predetermined period of time unless a cancel instruction is received, reversing movement of the motor to remove the suction tube, and deactivating the vacuum and switching to air.

44. A system according to embodiment 43 wherein depressing a first button results in a start instruction to be sent and depressing a second button results in a cancel instruction being sent.

45. A method for automated suctioning comprising:
a. activating a positioning system;
b. deploying a tube to a predetermined depth via the positioning system;
c. activating a suctioning means for a preset period of time;
d. deactivating said suctioning means following the expiration of said preset period of time;
e. activating said poristioning system; and
f. removing said tube via said positioning system.

46. The method of embodiment 45, wherein the positioning system is a motor and the motor is activated via one or more buttons or via eye tracking equipment.

47. The method of embodiment 45 or 46, wherein the suctioning means comprises a vacuum.

48. The method of embodiments 45-47, wherein the tube comprises a catheter.

1. A device comprising: an adaptor comprising: a first open-ended port having a proximal end configured to connect to a cannula and a distal end configured to connect to a ventilator tube; and, a second open-ended port for receiving a suctioning tube and guiding the suctioning tube into the cannula.

2. A device according to claim 1, wherein the adaptor is a universal adaptor compatible with existing ventilator systems.

3. A device according to claim 1, wherein the adaptor further comprises componentry for automated engagement and disengagement of the suctioning tube.

4. A device according to claim 3, further comprising a positioning system for positioning the suctioning tube by either deploying the suctioning tube into the cannula, removing it from the cannula, or both.

5. A device according to claim 4, wherein the positioning system is a motorized system that deploys the suctioning tube from a start position to an end position, which is in or though the cannula and that also retracts the suctioning tube to the start position.

6. A device according to claim 4, wherein the positioning system is a pneumatic system that deploys the suctioning tube from a start position to an end position, which is in or though the cannula and that also retracts the suctioning tube to the start position.

7. A system, comprising: a device according to claim 4; and, a mechanism for activating the suctioning tube positioning system.

8. A system according to claim 7, further comprising a suctioning tube and a suctioning device, which device creates suction in the suctioning tube when engaged.

9. A system according to claim 8, wherein the activation mechanism results in deploying the suctioning tube, activating suction, and thereafter removing the suctioning tube from the tracheostomy cannula.

10. A system according to claim 9, further comprising a mechanism for deactivating the activating mechanism to cancel deployment of the suctioning tube, suctioning, or both.

11. A system according to claim 10, wherein the deactivating mechanism further results in removing the suctioning tube from the cannula if it has been deployed in the cannula.

12. A system according to any of claim 7, further comprising a computer processing unit (“CPU”); a memory containing instructions for execution by the processor, which if executed results in automated control of the activation mechanism; and, a client user interface for triggering the processor to execute the instructions, wherein the client user interface comprises a button, which when depressed results in the processor executing the instructions, and wherein the instructions result in: activating the positioning system to deploy the suctioning tube into or through the cannula; activating the suctioning system to suction for an amount of time or until an amount of liquid is collected; and, activating the positioning system to remove the suctioning tube.

13. (canceled)

14. (canceled)

15. A system according to claim 12 wherein the positioning system deploys the suctioning tube by moving the suctioning tube in a forward direction through the port a predetermined distance or for a predetermined time, and removes the suctioning tube by moving the suctioning tube in a reverse direction for the same predetermined distance or predetermined time.

16. (canceled)

17. A system according to claim 15, further comprising a cancel button for shutting down deployment, suctioning or both, and wherein depressing the cancel button further results in retracting the suctioning tube to the start position.

18. (canceled)

19. A system according claim 7, wherein the system further comprises a computer program product for operating the suctioning system, comprising: a tangible computer readable storage medium having a computer readable program code embedded therein, the computer readable program code configured to: activate and deactivate the positioning system and activate and deactivate the suctioning system.

20. A system according to claim 19, wherein the positioning system is a motorized system and wherein activating and deactivating the positioning system and activating and deactivating the suctioning system comprises, when a start instruction is received, activating a vacuum and switching from air, running a motor for a predetermined period of time sufficient to deploy the suction tube unless a cancel instruction is received, thereafter operating or continuing to operate the vacuum for a predetermined period of time unless a cancel instruction is received, reversing movement of the motor to remove the suction tube, and deactivating the vacuum and switching to air.

21. A system according to claim 20 wherein depressing a first button results in a start instruction to be sent and depressing a second button results in a cancel instruction being sent.

22. A method for automated suctioning comprising:
a. activating a positioning system;
b. deploying a tube to a predetermined depth via the positioning system;
c. activating a suctioning system for a preset period of time;
d. deactivating said suctioning system following the expiration of said preset period of time;
e. activating said positioning system; and
f. removing said tube via said positioning system.

23. The method of claim 22, wherein the positioning system is a motor and the motor is activated via one or more buttons or via eye tracking equipment.

24. The method of claim 22, wherein the suctioning system comprises a vacuum.

25. The method of claim 22, wherein the tube comprises a catheter.
